(19) United States
(12) Patent Application Publication
(10) Pub. No.: US 2014/0046225 A1
(43) Pub. Date: Feb. 13, 2014

(54) FOCUSED ULTRASOUND THERAPY APPARATUS AND FOCAL POINT CONTROLLING METHOD THEREOF

(71) Applicant: Samsung Electronics Co., Ltd., Suwon-si (KR)

(72) Inventors: Jun-ho PARK, Hwaseong-si (KR); Sang-hyun Kim, Hwaseong-si (KR); Ho-Taik Lee, Yongin-si (KR)

(21) Appl. No.: 13/913,889

(22) Filed: Jun. 10, 2013

(30) Foreign Application Priority Data
Aug. 7, 2012 (KR) 10-2012-0086392

(51) Int. Cl. A61N 7/02

(52) U.S. Cl. CPC A61N 7/02
USPC 601/3

(57) ABSTRACT

A focal point controlling method of a focused ultrasound therapy apparatus includes receiving a plurality of focal point positions on which a focal point of an ultrasonic wave is to be formed, and receiving a sound pressure that is to be applied by the ultrasonic wave to each of the plurality of focal point positions, determining a particle velocity at each of a plurality of elements included in an ultrasound transducer, and radiating the ultrasonic wave according to the determined particle velocity. The particle velocity may be determined based on the received sound pressure that is to be applied by the ultrasonic wave to each of the plurality of focal point positions, by reflecting a sound pressure distribution of the ultrasonic wave that is generated by each of the plurality of elements.

### CROSS-REFERENCE TO RELATED APPLICATIONS

This application claims the priority benefit of Korean Patent Application No. 10-2012-0086392, filed on Aug. 7, 2012, in the Korean Intellectual Property Office, the disclosure of which is incorporated herein in its entirety by reference.

### BACKGROUND

1. Field

The disclosure herein relates to a focal point controlling method of a focused ultrasound therapy apparatus that removes a lesion by focusing ultrasonic waves.

2. Description of the Related Art

Along with the progress of medical science, recently, noninvasive surgery as well as minimum invasive surgery has been used for the local treatment of a tumor. Among the noninvasive surgery methods, high intensity focused ultrasound (HIFU) has become more widely used since ultrasound is generally harmless to the human body. HIFU therapy is a treatment method of necrotizing a lesion by focusing and radiating high intensity ultrasound to the lesion in the human body. An ultrasound which is focused and radiated on the lesion is converted into thermal energy that causes coagulating necrosis of the lesion and blood vessels due to a temperature increase of a portion of the lesion to which the ultrasound is radiated. Since the temperature is raised instantly, it is possible to effectively remove only the radiated portion while preventing heat from diffusing to surrounding areas of the radiated portion.

A focused ultrasound therapy apparatus may include a transducer for transducing an electric signal into an ultrasonic wave and may control a position at which a focal point is formed by adjusting a particle velocity at the transducer. Recently, a method of simultaneously forming focal points on a plurality of focal point positions by using an ultrasound transducer including a plurality of elements has been used. However, to minimize an influence on surrounding tissues and remove only a target lesion when simultaneously forming focal points on a plurality of focal point positions, surround pressures, which are applied to the plurality of focal point positions, need to be uniform within a predetermined range. Further, temperatures of the plurality of focal point positions need to be uniform within a predetermined range.

### SUMMARY

Disclosed herein are focal point controlling methods of a focused ultrasound therapy apparatus, which uniformly control sound pressures, which are applied to a plurality of focal point positions, and temperatures of the plurality of focal point positions.

Additional aspects will be set forth in part in the description which follows and, in part, will be apparent from the description, or may be learned by practice of the presented embodiments.

According to an aspect of the present invention, a focal point controlling method of a focused ultrasound therapy apparatus includes: receiving a plurality of focal point positions on which a focal point of an ultrasonic wave is to be formed, and receiving a sound pressure that is to be applied by the ultrasonic wave to each of the plurality of focal point positions; determining a particle velocity at each of a plurality of elements included in an ultrasound transducer, which is required to apply the received sound pressure to each of the plurality of focal point positions, by reflecting a sound pressure distribution of the ultrasonic wave that is generated by each of the plurality of elements; and radiating the ultrasonic wave according to the determined particle velocity.

The determining of the particle velocity maybe performed by calculating, with respect to the plurality of elements and the plurality of focal point positions, a factor indicating the degree in which an ultrasonic wave generated by any one element has an influence on a sound pressure which is applied to any one focal point position; reflecting the calculated factor to a characteristic equation that indicates a relation between the particle velocity at any one element and the sound pressure which is applied to any one focal point position; and determining the particle velocity at each of the plurality of elements by using the characteristic equation in which the factor has been reflected.

The value of the factor may have a form of a Gaussian distribution according to an angle between a straight line, which is obtained by connecting a center of gravity of an element with a focal point position, and a cross section in which an ultrasonic wave of the element is generated. The factor may have a value in the range of 0 to 1. When the value of the factor is 1, a focal point position may exist on a straight line that is perpendicular to a cross section in which an ultrasonic wave of the element is generated, from which the ultrasonic wave of the element is generated, and passes through a center of gravity of the element. When the value of the factor is 0, the focal point position may exist on a plane that is obtained by extending the cross section. The factor may be calculated with respect to each of the plurality of focal point positions at each of the plurality of elements.

The focal point controlling method may further include measuring a temperature of each of the plurality of focal point positions, wherein the particle velocity at each of the plurality of elements included in the ultrasound transducer may be determined based on the temperatures of the plurality of focal point positions. The particle velocity may be determined by determining whether a temperature difference between the plurality of focal point positions is equal to or greater than a predetermined critical value, and if the temperature difference is equal to or greater than the predetermined critical value, the particle velocity may be determined so that the temperature difference between the plurality of focal point positions is less than the predetermined critical value.

According to another aspect of the present invention, a focused ultrasound therapy apparatus includes: an ultrasound transducer that transduces an electrical signal into an ultrasonic wave; an input unit that receives a plurality of focal point positions on which a focal point of an ultrasonic wave is to be formed, and a sound pressure that is to be applied by the ultrasonic wave to each of the plurality of focal point positions; and a focal point controller that determines a particle velocity at each of a plurality of elements included in the ultrasound transducer, which is required to apply the received sound pressure to each of the plurality of focal point positions, by reflecting a sound pressure distribution of the ultrasonic wave generated by each of the plurality of elements and controls the ultrasound transducer.

The focal point controller may include: a factor calculator that calculates, with respect to the plurality of elements and the plurality of focal point positions, a factor indicating the degree in which an ultrasonic wave generated by any one element has an influence on a sound pressure which is applied to any one focal point position; and a particle velocity determination unit that determines the particle velocity by reflecting the calculated factor to a characteristic equation which indicates a relation between the particle velocity at any one element and the sound pressure which is applied to any one focal point position.

The factor may have a form of a Gaussian distribution according to an angle between a straight line, which is obtained by connecting a center of gravity of an element with a focal point position, and a cross section in which an ultrasonic wave of the element is generated. The factor may have a value in the range of 0 to 1. The value of the factor may be 1 when a focal point position exists on a straight line that is perpendicular to a cross section in which an ultrasonic wave of the element is generated, from which the ultrasonic wave of the element is generated, and passes through a center of gravity of the element. The value of the factor may be 0 when the focal point position exists on a plane that is obtained by extending the cross section. The factor calculator may calculate a factor with respect to each of the plurality of focal point positions at each of the plurality of elements.

The focused ultrasound therapy apparatus may further include a feedback unit to measure a temperature of each of the plurality of focal point positions and to feed the measured temperature back to the focal point controller to adjust a particle velocity so that the temperatures of the plurality of focal point positions become uniform. The feedback unit may determine whether a temperature difference between the plurality of focal point positions is equal to or greater than a predetermined critical value, and if the temperature difference is equal to or greater than the predetermined critical value, the focal point controller may adjust the particle velocity to make the temperature difference between the plurality of focal point positions be less than the predetermined critical value.

According to an embodiment of the present invention a focal point controlling method of a focused ultrasound therapy apparatus may include controlling sound pressures to be applied to a plurality of focal point positions on which a focal point of an ultrasonic wave is to be formed by determining a particle velocity at each of a plurality of elements included in an ultrasound transducer through reflecting a sound pressure distribution of the ultrasonic wave generated by each of the plurality of elements; and radiating the ultrasonic wave according to the determined particle velocity. The focal point controlling method may further include controlling temperatures of the plurality of focal point positions on which the focal point of the ultrasonic wave is formed by determining whether a temperature difference between the plurality of focal point positions exceeds a threshold value and by adjusting a particle velocity for each of the plurality of elements based on the determination.

According to an embodiment of the present invention a non-transitory computer-readable recording medium may have recorded thereon a program for executing the focal point controlling methods disclosed herein.

According to an embodiment of the present invention, sound pressures that are applied to a plurality of focal point positions may be uniformly controlled by calculating a particle velocity at each element by reflecting a sound pressure distribution of an ultrasound that is generated in each of a plurality of elements of an ultrasound transducer.

In addition, the temperatures of the plurality of focal point positions may be uniformly controlled by monitoring the temperature of each focal point position while performing a focused ultrasound therapy and feeding the monitored temperature back.

### BRIEF DESCRIPTION OF THE DRAWINGS

These and/or other aspects will become apparent and more readily appreciated from the following description of the embodiments, taken in conjunction with the accompanying drawings in which:

FIG. 1 is a diagram illustrating a focused ultrasound therapy apparatus according to an embodiment of the present invention;

FIG. 2 is a diagram illustrating a position vector for any one element included in an ultrasound transducer illustrated in FIG. 1 and a position vector for a focal point position;

FIG. 3 is a diagram that illustrates an element and a position vector of a focal point position according to an embodiment of the present invention;

FIGS. 4A and 4B are diagrams each illustrating a sound pressure distribution of an ultrasonic wave that is generated by one element, according to an embodiment of the present invention;

FIG. 5 is a diagram that illustrates an n-th element and a position vector of an m-th focal point position according to an embodiment of the present invention;

FIG. 6 is a diagram illustrating a focused ultrasound therapy apparatus according to another embodiment of the present invention; and

FIGS. 7 and 8 are flowcharts each illustrating a focal point controlling method of a focused ultrasound therapy apparatus, according to an embodiment of the present invention.

### DETAILED DESCRIPTION

Exemplary embodiments of the present invention will now be described more fully with reference to the accompanying drawings, wherein like reference numerals refer to like elements throughout. In the following description, well-known functions or constructions are not described in detail if it is determined that they would obscure the invention due to unnecessary detail.

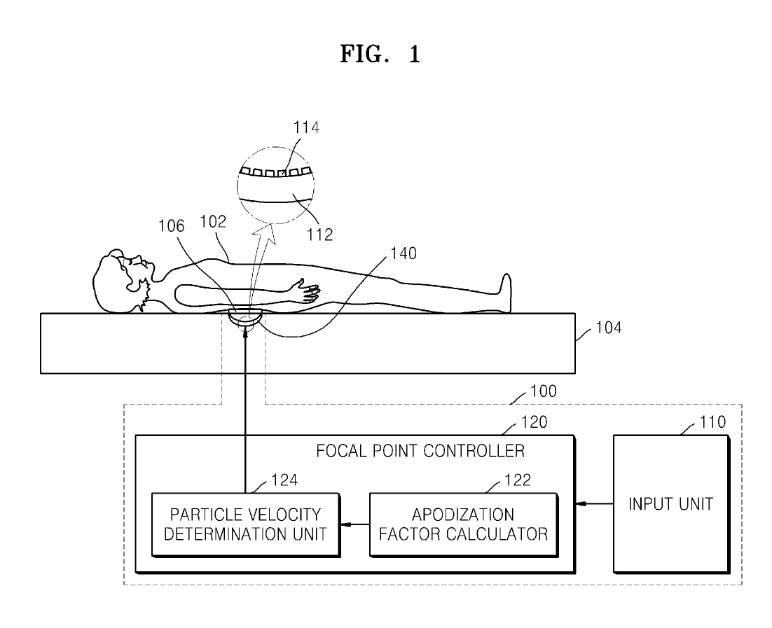

FIG. 1 is a diagram illustrating a focused ultrasound therapy apparatus 100 according to an embodiment of the present invention. Referring to FIG. 1, the focused ultrasound therapy apparatus 100 may include an input unit 110, a focal point controller 120, and an ultrasound transducer 140, wherein the focal point controller 120 may include an apodization factor calculator 122 and a particle velocity determination unit 124.

As illustrated in FIG. 1, the ultrasound transducer 140 may be installed inside a bed 104 on which a subject 102 to be diagnosed is lying, and may remove a lesion by radiating an ultrasonic wave to a predetermined portion of a body of the subject 102. Here, a gel pad 106 may be positioned between the subject 102 and the table or bed 104 to help the transmission of the ultrasonic wave.

Before describing an operation of each component of the focused ultrasound therapy apparatus 100, a method of controlling a focal point of an ultrasonic wave that is radiated from the ultrasound transducer 140 is described.

As shown in a magnified figure in FIG. 1, the ultrasound transducer 140 may include a plurality of elements 114 for generating the ultrasonic wave on a round-type support plate 112 concave at the center. For example, the support plate 112 may be semi-circular or arc-shaped, as shown in FIG. 1 and the plurality of elements 114 may be arranged at a center portion of the support plate 112. However, the support plate 112 need not be rounded or arc-shaped, and may be of another shape. For example, the support plate 112 may be rectangular in shape or another polygonal or geometric shape, and the plurality of elements 114 may be arranged on a portion of the support plate 112. By controlling a particle velocity of the ultrasonic wave at each of the plurality of elements 114, a position at which a focal point of the ultrasonic wave is formed and a sound pressure may be controlled. The particle velocity of the ultrasonic wave indicates an amplitude and phase of the ultrasonic wave that is generated by each of the plurality of elements 114.

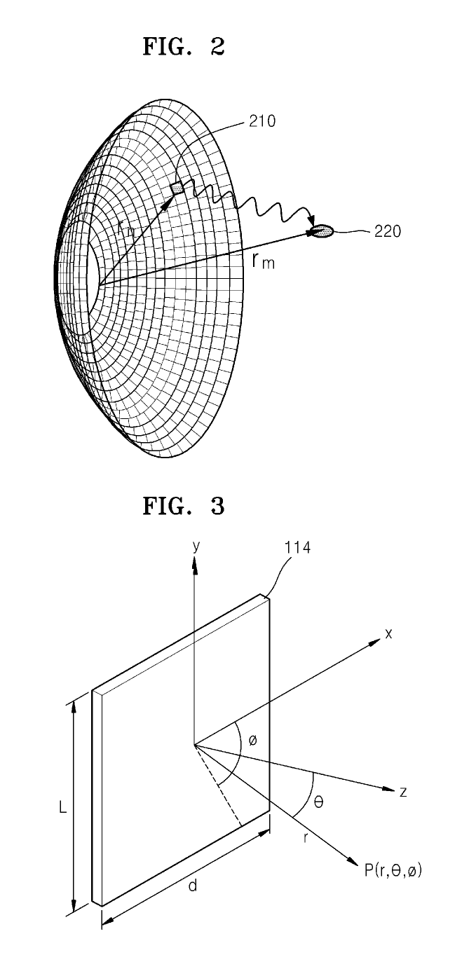

For example, a case in which the ultrasound transducer 140 includes N elements and a focal point is formed on each of M target positions is described below (where N and M are natural numbers). FIG. 2 is a diagram illustrating a position vector for an element included in the ultrasound transducer 140 and a position vector for a focal point position. Referring to FIG. 2, “rₙ” is a position vector of an n-th element 210 (where n is 1, 2, . . . , or N), and “rₘ” is a position vector of an m-th target position 220 (where m is 1, 2, . . . , or M). In this case, a sound pressure “p” that is applied to the m-th target position 220 by the N elements (represented by the squiggly arrowed-line drawn from the n-th element 210 to the m-th target position 220) may be obtained by using a Rayleigh-Sommerfeld integral that is represented as the following Equation 1.

$$\frac{j ρ ck}{2 π} {\sum }_{n = 1}^{N} {U}_{n} {\int }_{{S}_{n}} \frac{{e}^{- j k \vert {r}_{m} - {r}_{n} \vert}}{\vert {r}_{m} - {r}_{n} \vert} d {S}_{n} = p ( {r}_{m} ) \qquad ( 1 )$$

In Equation 1, “p”, “c”, and “k” indicate a density of a uniform tissue, a propagation velocity of an ultrasonic wave in the tissue, and a wave number of the ultrasonic wave, respectively. “Sₙ,” is a cross-sectional area of the n-th element 210. “uₙ” is a particle velocity at the n-th element 210, “p(rₘ)” is a sound pressure at a target position having the position vector “rₘ”. Based on Equation 1, a relation equation (that is, ultrasonic wave propagation characteristics) between a particle velocity at the n-th element 210 and a sound pressure that is applied to the m-th target position 220 may be obtained as the following Equation 2.

$$H ( m , n ) = \frac{j ρ ck}{2 π} {\int }_{{S}_{n}}^{ } \frac{{e}^{- j k \vert {r}_{m} - {r}_{n} \vert}}{\vert {r}_{m} - {r}_{n} \vert} d {S}_{n} \qquad ( 2 )$$

Based on Equations 1 and 2, a relation equation with respect to a matrix “u” for particle velocities at the N elements, a matrix “p” for sound pressures that are applied to the M target positions, and a matrix “H” for ultrasonic wave propagation characteristics may be obtained as the following Equation 3.

$$[ \begin{matrix}{H}_{( 1 , 1 )} & {H}_{( 1 , 2 )} & {H}_{( 1 , 3 )} & … & {H}_{( 1 , n )} \\ {H}_{( 2 , 1 )} & {H}_{( 2 , 2 )} & {H}_{( 2 , 3 )} & … & {H}_{( 2 , n )} \\ ⋮ & ⋮ & ⋮ & ⋮ & ⋮ \\ {H}_{( m , 1 )} & {H}_{( m , 2 )} & {H}_{( m , 3 )} & … & {H}_{( M , N )}\end{matrix} ] [ \begin{matrix}{u}_{1} \\ {u}_{2} \\ ⋮ \\ {u}_{N}\end{matrix} ] = [ \begin{matrix}{p}_{1} \\ {p}_{2} \\ ⋮ \\ {p}_{M}\end{matrix} ] \qquad ( 3 )$$

A particle velocity at each element, which is required to apply a desired sound pressure, may be obtained by using a pseudoinverse method according to the following Equations 4 through 6.

Hu=p (4)

u=H⁺p (5)

u=H*^{t}(HH*^{t})⁻¹p (6)

In Equations 5 and 6, “H+” is a pseudoinverse matrix of “H”, and “H*^{t}” is a conjugate transpose matrix of “H”.

However, sound pressures that are applied to a plurality of focal point positions are not uniform when actually radiating an ultrasonic wave according to a particle velocity calculated by the method described above. This is because a sound pressure distribution of an ultrasonic wave that is generated by each element included in the ultrasound transducer 140 has the form of a Gaussian or sinc function. In more detail, since the edge of each element included in the ultrasound transducer 140 is fixed to the support plate 112 of the ultrasound transducer 140, a tremble in the center of each element is larger than that at the edge thereof while an ultrasonic wave is generated. Accordingly, the closer a point is to the center of each element, a stronger sound pressure may be applied thereto.

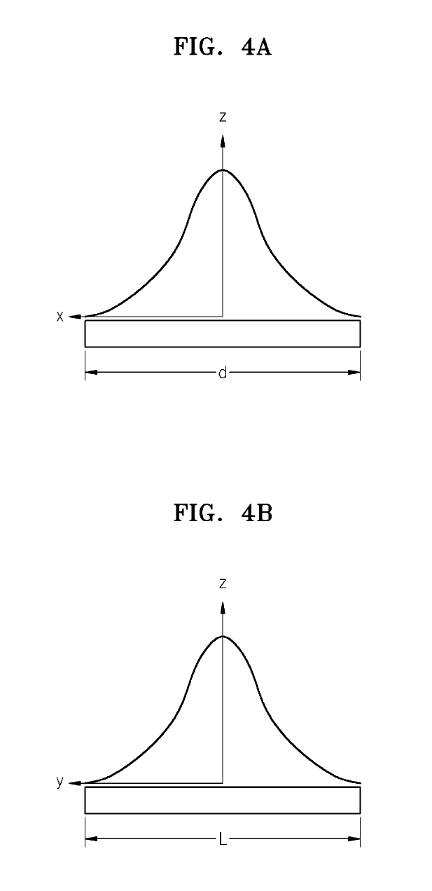

FIG. 3 shows a diagram that illustrates an element 114 and a position vector r of a focal point position according to an embodiment of the present invention. Referring to FIG. 3, “d” is the width of the element 114, “L” is the length of the element 114, and the z-axis direction is a direction in which an ultrasonic wave is radiated. Further, “θ” is an angle between the position vector “r” and the z-axis, and “φ” is an angle between a segment (shown by the dashed line) which is obtained by projecting the position vector “r” to the x-y plane, and the x-axis. Sound pressure “P” may be generated by element 114, and may be a function of “r,” “θ,” and “φ”. As shown in FIG. 3, the position vector “r” may be projected from a center of the element 114 toward the focal point position.

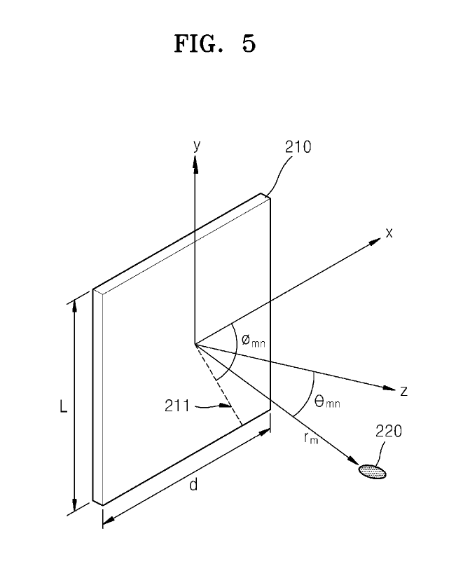

The sound pressure distribution of an ultrasonic wave that is generated by such an element is illustrated in FIG. 4A and FIG. 4B. A bell-shaped curve having the form of a Gaussian function, illustrated in FIG. 4A, is a curve obtained by connecting points, to which uniform sound pressures are applied due to an ultrasonic wave which is generated by the single n-th element 210, to each other. Here, the x-axis direction is a direction of the width of the n-th element 210, and “d” is the width of the n-th element. The z-axis direction is a direction in which an ultrasonic wave is radiated from the n-th element 210. A bell-shaped curve having the form of a Gaussian function, illustrated in FIG. 4B, is also a curve obtained by connecting points, to which uniform sound pressures are applied due to an ultrasonic wave which is generated by the single n-th element 210, to each other. Here, the y-axis direction is a direction of the length of the n-th element 210, and “L” is the length of the n-th element 210. The z-axis direction is a direction in which an ultrasonic wave is radiated from the n-th element 210.

As described above, in order to form uniform sound pressures, which are applied to a plurality of focal point positions, by minimizing an influence of the sound pressure distribution of each ultrasonic wave that is generated by each single element, a factor indicating the degree in which the ultrasonic wave generated by the single element has an influence on a sound pressure which is applied to any one focal point position is calculated and a particle velocity is determined by reflecting the calculated factor. In the current embodiment, such a factor is referred to as an apodization factor.

The apodization factor may be set to have, for example, a value in the range of 0 to 1. For example, an apodization factor of 1 may indicate that an ultrasonic wave, which is generated by an element, is 100 percent transmitted to a focal point position and thus is applied thereto as a sound pressure. On the contrary, an apodization factor of 0 may indicate that an ultrasonic wave, which is generated by an element, is not transmitted to a focal point position and thus is not applied thereto at all as a sound pressure. For example, the value of the apodization factor may be 1 when a focal point position exists on a straight line that is perpendicular to a cross section in which an ultrasonic wave of the element is generated, from which the ultrasonic wave of the element is generated, and passes through the center of gravity of the element, and the value of the factor may be 0 when the focal point position exists on a plane that is obtained by extending the cross section. An example method of calculating an apodization factor is described in detail below.

Disclosed herein is an operation of controlling a focal point by reflecting the apodization factor, which is performed in the focused ultrasound therapy apparatus 100 according to an embodiment of the present invention.

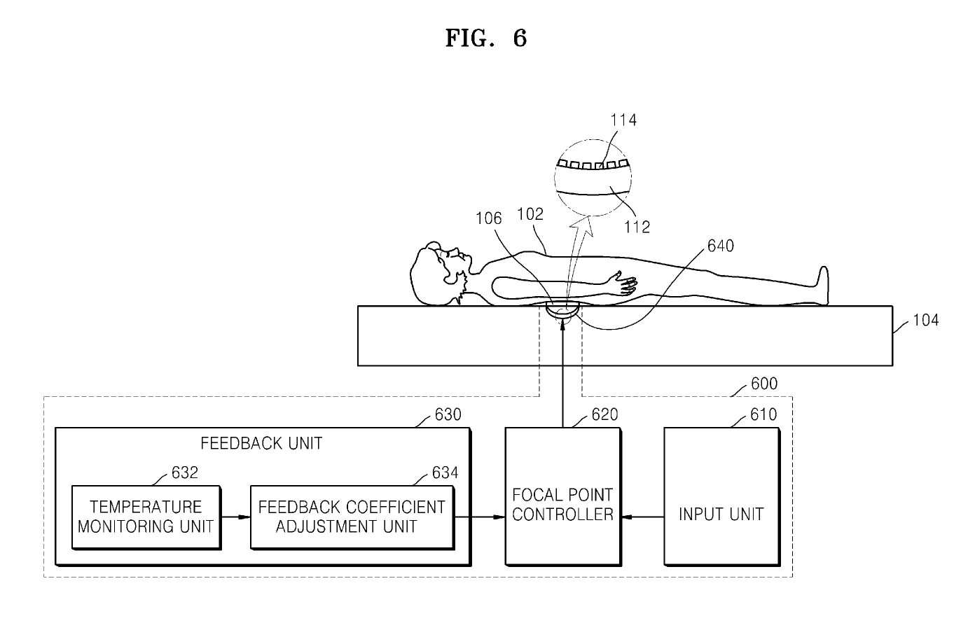

Referring back to FIG. 1, the input unit 110 receives, from a user, a plurality of focal point positions on which a focal point of an ultrasonic wave has to be formed to remove a lesion, and a sound pressure that has to be applied to each of the plurality of focal point positions by the ultrasonic wave. When the input unit 110 receives the plurality of focal point positions and the sound pressure, the apodization factor calculator 124 calculates each element's apodization factor for the received plurality of focal point positions. An n-th element's apodization factor for an m-th focal point position is referred to as “aₘₙ”, and a method of calculating “aₘₙ” is described in detail with reference to FIG. 5 below.

FIG. 5 is a diagram that illustrates an n-th element 210 and a position vector of an m-th focal point position 220. Referring to FIG. 5, “d” is the width of the n-th element 210, “L” is the length of the n-th element 210, and the z-axis direction is a direction in which an ultrasonic wave is radiated. “rₘ” is a position vector of the m-th focal point position 220, “θₘₙ,” is an angle between the position vector “rₘ” and the z-axis, and “φₘₙ,” is an angle between a segment 211, which is obtained by projecting the position vector “rₘ” to the x-y plane, and the x-axis. By way of example, the position vector “rₘ” may be projected from a center of the n-th element 210 toward the m-th focal point position 220. By way of example, the position vector “rₘ” may be projected from a center of gravity of the n-th element 210 toward the m-th focal point position 220. When the n-th element 210 and the m-th focal point position 220 are defined as in FIG. 5, the apodization factor “aₘₙ” may be calculated according to the following Equation 7.

$${a}_{mn} = \vert sin c ( \frac{π {d}_{n} sin {θ}_{mn} cos {φ}_{mn}}{λ} ) sin c ( \frac{π {L}_{n} sin {θ}_{mn} sin {φ}_{mn}}{λ} ) \vert \cdot k \qquad ( 7 )$$

In Equation 7, “k” is a scale factor, and a value of “k” may be obtained as follows. After setting “k” to an arbitrary value and forming a plurality of focal points by controlling the ultrasound transducer 140 according to Equations 7 and 8 and the following description, a sound pressure of each focal point is measured. The focal points are formed while varying the value of “k”, and then the sound pressure of each focal point is measured. Based on the measured sound pressures, a value of “k” when the sound pressures of the focal points are most uniform is determined to be a final value of “k”.

When the apodization factor calculator 122 obtains all of the n elements' apodization factors for m focal point positions according to Equation 7, the particle velocity determination unit 124 obtains ultrasonic wave propagation characteristics by reflecting the n elements'apodization factors. The n-th element 210's ultrasonic wave propagation characteristics for the m-th focal point position 220 to which an apodization factor has been reflected are represented as the following Equation 8.

$$H ( m , n ) = \frac{j ρ ck}{2 π} {\int }_{{S}_{n}}^{ } \frac{{e}^{- j k \vert {r}_{m} - {r}_{n} \vert}}{\vert {r}_{m} - {r}_{n} \vert} d {S}_{n} \cdot {a}_{mn} \qquad ( 8 )$$

Equation 8 further includes the apodization factor “aₘₙ” compared to Equation 2 that represents ultrasonic wave propagation characteristics which are used in a general focal point controlling method. After obtaining the ultrasonic wave propagation characteristics according to Equation 8, the particle velocity determination unit 124 determines a particle velocity at each element, which is required to apply a desired sound pressure, by using the pseudoinverse method according to Equations 4 through 6, thereby controlling the ultrasound transducer 140.

In this manner, by reflecting each element's apodization factor for a target focal point position when determining a particle velocity at each element, it is possible to control a focal point so that a desired surround pressure is accurately applied to the target focal point position. That is, a correct lesion may be removed by removing sound pressure non-uniformity between focal point positions, which occurs due to the sound pressure distribution of an ultrasonic wave that is generated by each element. That is, a lesion may be removed by ensuring a uniform sound pressure being applied to each focal point position, caused by ultrasonic waves generated from corresponding elements.

Although uniform sound pressure is applied to a plurality of focal point positions by using the above focal point controlling method, the temperatures of the plurality of focal point positions may not be uniform due to various factors, such as the properties of tissue of a portion on which a focal point is formed, the properties of tissue on a path through which an ultrasonic wave is transmitted, or the like. Accordingly, to uniformly control the temperatures of the plurality of focal point positions, it is necessary to monitor the temperature while performing the radiation of an ultrasonic wave and then feed the monitored temperature back.

A focused ultrasound therapy apparatus according to another embodiment of the present invention is described below. Here, to uniformly control the temperatures of the plurality of focal point positions, a function of monitoring the temperatures and then feeding the monitored temperatures back is additionally provided.

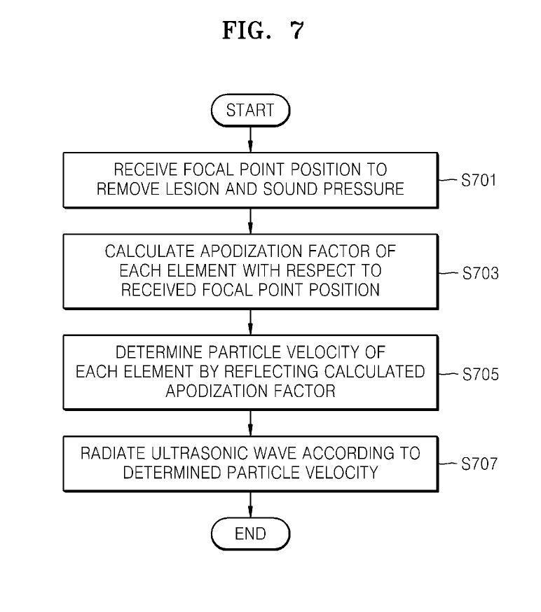

FIG. 6 is a diagram illustrating a focused ultrasound therapy apparatus 600 according to another embodiment of the present invention. Referring to FIG. 6, the focused ultrasound therapy apparatus 600 may include an input unit 610, a focal point controller 620, a feedback unit 630, and an ultrasound transducer 640, wherein the feedback unit 630 may include a temperature monitoring unit 632 and a feedback coefficient adjustment unit 634.

The input unit 610 of the focused ultrasound therapy apparatus 600 performs the same operation as the input unit 110 of the focused ultrasound therapy apparatus 100 illustrated in FIG. 1, and thus, a detailed description thereof is omitted.

As shown in the following Equation 9, the focal point controller 620 of the focused ultrasound therapy apparatus 600 adds a feedback coefficient w to an ultrasonic wave propagation characteristic relation equation and then determines a particle velocity. The feedback coefficient w is a coefficient that is adjusted to uniformly control the temperatures of a plurality of focal point positions and may be initially set to a predetermined value, for example, “1”.

$$[ \begin{matrix}{H}_{( 1 , 1 )} & {H}_{( 1 , 2 )} & {H}_{( 1 , 3 )} & … & {H}_{( 1 , n )} \\ {H}_{( 2 , 1 )} & {H}_{( 2 , 2 )} & {H}_{( 2 , 3 )} & … & {H}_{( 2 , n )} \\ ⋮ & ⋮ & ⋮ & ⋮ & ⋮ \\ {H}_{( m , 1 )} & {H}_{( m , 2 )} & {H}_{( m , 3 )} & … & {H}_{( M , N )}\end{matrix} ] [ \begin{matrix}{u}_{1} \\ {u}_{2} \\ ⋮ \\ {u}_{N}\end{matrix} ] = [ \begin{matrix}{W}_{1} & 0 & 0 & … & 0 \\ 0 & {W}_{2} & 0 & … & 0 \\ ⋮ & ⋮ & ⋮ & ⋮ & ⋮ \\ 0 & 0 & 0 & … & {W}_{M}\end{matrix} ] [ \begin{matrix}{P}_{1} \\ {P}_{2} \\ ⋮ \\ {P}_{M}\end{matrix} ] \qquad ( 9 )$$

The focal point controller 620 may determine a particle velocity by performing a pseudoinverse method according to the following Equations 10 through 12. In this case, an ultrasonic wave propagation characteristic matrix H may be obtained by Equations 7 and 8.

Hu=wp (10)

u=H⁺wp (11)

u=H*^{t}(HH*^{t})⁻¹wp (12)

As described above, the feedback unit 630 may include the temperature monitoring unit 632 and the feedback coefficient adjustment unit 634. The temperature monitoring unit 632 monitors a temperature of a position on which a focal point is formed while performing a focused ultrasound therapy. The feedback coefficient adjustment unit 634 adjusts a feedback coefficient based on the monitored temperature and provides the adjusted feedback coefficient to the focal point controller 620. The focal point controller 620 determines a particle velocity at each element of the ultrasound transducer 640 by reflecting the adjusted feedback coefficient, and then controls a focal point. For example, when a temperature difference between a plurality of focal point positions is equal to or greater than a predetermined critical value, the feedback coefficient adjustment unit 634 adjusts a feedback coefficient, which corresponds to a focal point position of which the temperature is relatively high, to less than “1” and adjusts a feedback coefficient, which corresponds to a focal point position of which the temperature is relatively low, to more than “1”. For example, if a temperature difference between a first focal point position and a second focal point position is greater than a predetermined critical value, the feedback coefficient may be adjusted. If the temperature at the focal point position for which a feedback coefficient is to be adjusted (for example, the first focal point position) is relatively high, compared to a predetermined threshold, the feedback coefficient may be set to be a value less than one. On the other hand, if the temperature at the first focal point is relatively low, compared to a predetermined threshold, the feedback coefficient may be set to be a value more than one. When the feedback coefficients are calculated, the focal point controller 620 determines a particle velocity at each element by reflecting the calculated feedback coefficients.

In this manner, by monitoring the temperature of a position, on which a focal point of an ultrasonic wave is formed while performing a focused ultrasound therapy, and feeding the monitored temperature back, a treatment area on which an ultrasonic wave is radiated may be controlled with a desired temperature. Furthermore, a correct lesion may be removed by solving a temperature non-uniformity between focal point positions, which may occur due to various factors.

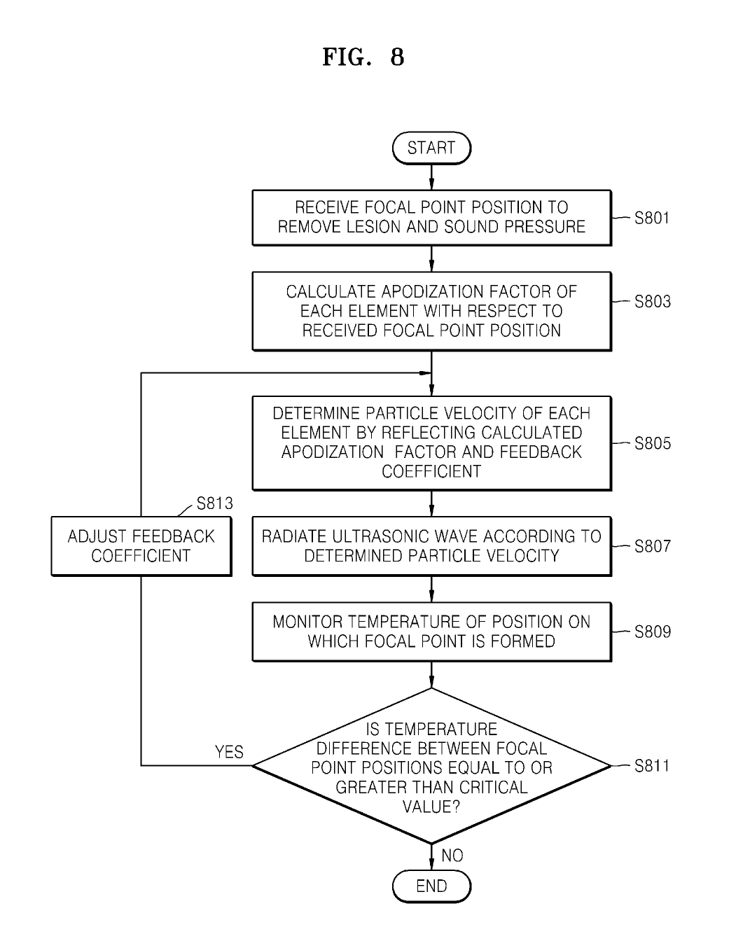

Below, a focal point controlling method of a focused ultrasound therapy apparatus according to an embodiment of the present invention is described. FIGS. 7 and 8 are flowcharts each illustrating a focal point controlling method of a focused ultrasound therapy apparatus, according to an embodiment of the present invention. In particular, FIG. 7 relates to a focal point controlling method that includes calculating each element's apodization factor for a plurality of focal point positions and then determining a particle velocity of each element by reflecting the calculated apodization factor. FIG. 8 relates to a focal point controlling method that further includes monitoring the temperature of a focal point position and feeding the monitored temperature back, compared to the focal point controlling method of FIG. 7.

Referring to FIG. 7, in order to perform a focused ultrasound therapy, in operation S701, a focal point position on which a focal point has to be formed to remove a lesion and a sound pressure that has to be applied to the focal point position is received from a user. In particular, images of the internal tissues of the body, which has a lesion, may be displayed to a user and a coordinate value corresponding to the focal point position may be received from the user. Alternatively, images of the internal tissues of the body may be displayed on a display unit supporting a touch function and the focal point position may be received through a touch input. Here, although two methods are described as examples, the present invention is not limited thereto and the focal point position may be received through various methods that are generally used.

In operation S703, an apodization factor of each element of the ultrasound transducer with respect to the received focal point position is calculated. If a plurality of focal point positions are received in operation S701 and the ultrasound transducer includes a plurality of elements, all apodization factors of the plurality of elements with respect to each of the received plurality of focal point positions are calculated. If it is assumed that M focal point positions are set and the ultrasound transducer includes N elements, an example method of calculating an apodization factor is disclosed above in which a process in which the apodization factor calculator 122 calculates an apodization factor is described with reference to FIG. 1, FIG. 5, and Equation 7.

Subsequently, in operation S705, a particle velocity of each element of the ultrasound transducer is calculated reflecting the apodization factor calculated in operation S703. An example method of determining a particle velocity by multiplying an apodization factor to an ultrasonic propagation characteristic equation and using the pseudoinverse method is disclosed above with reference to Equations 4 through 6 and Equation 9.

Finally, in operation S707, a focused ultrasound therapy is performed by radiating an ultrasonic wave according to a particle velocity determined in operation S705.

In this manner, when determining a particle velocity at each element of the ultrasound transducer, by reflecting each element's apodization factor for a target focal point position, it is possible to control a focal point so that a desired sound pressure is accurately applied to the target focal point position. That is, a correct lesion may be removed by removing sound pressure non-uniformity between focal point positions, which occurs due to the sound distribution of an ultrasonic wave that is generated by any one element.

A focal point controlling method that further includes monitoring the temperature of a focal point position and feeding the monitored temperature back is described with reference to FIG. 8. Referring to FIG. 8, operations S801 through S807 are similar to operations S701 through S707 illustrated in FIG. 7, and thus, a detailed description of operations S801 through S807 is omitted. However, operation S805 is different from operation S705 in that, in operation S805, a particle velocity is determined by reflecting a feedback coefficient as well as an apodization factor. An example method of determining a particle velocity by reflecting a feedback coefficient is disclosed above with reference to Equations 9 through 12.

In operation S809, the temperature of a position on which a focal point is formed is monitored while performing a focused ultrasound therapy. In operation S811, it is determined whether a temperature difference between a plurality of focal point positions is equal to or greater than a predetermined critical value. If the temperature difference is equal to or greater than the predetermined critical value, operation S813 is performed to adjust the feedback coefficient. For example, if a temperature difference between a plurality of focal point positions is equal to or greater than a predetermined critical value, a feedback coefficient, which corresponds to a focal point position of which the temperature is relatively high, is adjusted to be less than a predetermined value (for example, “1”) and a feedback coefficient, which corresponds to a focal point position of which the temperature is relatively low, is adjusted to more than a predetermined value (for example, “1”).

When the feedback coefficient is adjusted in operation S813, the method returns to operation S805 and, a particle velocity of each element of the ultrasound transducer is calculated by reflecting the apodization factor calculated in operation S803 and the feedback coefficient adjusted in operation S813. An example method of determining a particle velocity by reflecting an apodization factor and a feedback coefficient is disclosed above with reference Equations 7 through 12.

In this manner, by monitoring the temperature of a position, on which a focal point of an ultrasonic wave is formed while performing a focused ultrasound therapy, and feeding the monitored temperature back, the temperatures of a plurality of focal point positions may be uniformly controlled, and thus, an influence on surrounding tissue may be minimized and only a lesion may be accurately removed.

The disclosure herein has described one or more embodiments in which a focused ultrasound therapy apparatus and focal controlling method may be used to treat a patient, such as a human having a lesion needing to be removed. However, the focused ultrasound therapy apparatus and focal controlling method may be used in the treatment of other life forms, including animals. Additionally, it should be noted that while FIGS. 1 and 4 illustrate treatment of a lesion using an ultrasonic transducer while a subject lays on a bed or table, the disclosure is not so limited. For example, the subject may be treated while in another position, the ultrasonic transducer may be disposed elsewhere to treat another area of the subject, there may not be a bed, or there may be another object in which the ultrasonic transducer is installed, or the ultrasonic transducer may be portable.

In one or more previously described embodiments, it has been disclosed that the input unit receives, from a user, a plurality of focal point positions on which a focal point of an ultrasonic wave has to be formed to remove a lesion, and a sound pressure that has to be applied to each of the plurality of focal point positions by the ultrasonic wave. The focal point positions and sound pressure information may be received from the user via a plurality of methods. For example, the focal point positions and sound pressure information may be obtained by the input unit via a wired or wireless network, or from a non-transitory computer-readable media including magnetic media such as hard disks, floppy disks, and magnetic tape; optical media such as CD ROM disks and DVDs; magneto-optical media such as optical disks; or from other hardware devices that are configured to store data, such as a USB or flash memory.

In one or more previously described embodiments, it has also been disclosed that the ultrasound transducer includes a plurality of elements arranged on a support plate. The shape and size of the plurality of elements may be varied. For example, the plurality of elements may be rectangular, square, circular, triangular, polygonal, or any other geometric shape. The plurality of elements may be uniform in shape and/or size or different in shape and/or size from one another.

In one or more previously described embodiments, it has also been disclosed that the focused ultrasound therapy apparatus includes a feedback unit which monitors the temperature of a focal point and a plurality of focal point positions while performing a focused ultrasound therapy. A feedback coefficient may be adjusted based on the monitored temperatures and a particle velocity at each element of the ultrasound transducer may be determined by reflecting the adjusted feedback coefficient, to control a focal point. The feedback coefficient may be adjusted to be greater than one or less than one, depending on whether a temperature difference between the plurality of focal point positions is equal to or greater than a threshold value, and depending on whether the temperature of the focal point position is relatively high or relatively low. One of ordinary skill in the art would understand that a lookup table may be employed to determine the appropriate feedback coefficient based on the measured temperature and the properties of the treatment area being treated. For example, a desired or safe temperature value for an operation on a particular tissue type being treated may be determined beforehand, and a coefficient may be obtained from the lookup table based on a measured temperature. For example, if a first measured temperature exceeds a desired temperature (or acceptable temperature range) for that tissue type, then an obtained first coefficient may be a value less than one. If a second measured temperature is higher than the first measured temperature, the obtained second coefficient may be a value lower than the first coefficient. A similar rationale may be applied to temperatures which are less than the desired temperature or range of temperature for a tissue type. The coefficients are reflected into the calculation for the particle velocity, which affects the temperature of the focal point corresponding to the lesion being treated.

The terms “module”, and “unit,” as used herein, may refer to, but is not limited to, a software or hardware component or device, such as a Field Programmable Gate Array (FPGA) or Application Specific Integrated Circuit (ASIC), which performs certain tasks. A module or unit may be configured to reside on an addressable storage medium and configured to execute on one or more processors. Thus, a module or unit may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules/units may be combined into fewer components and modules/units or further separated into additional components and modules.

The focused ultrasound therapy apparatus and focal point controlling method according to the above-described example embodiments may use one or more processors, which may include a microprocessor, central processing unit (CPU), digital signal processor (DSP), or application-specific integrated circuit (ASIC), as well as portions or combinations of these and other processing devices.

The focal point controlling method according to the above-described example embodiments may be recorded in non-transitory computer-readable media including program instructions to implement various operations embodied by a computer. The media may also include, alone or in combination with the program instructions, data files, data structures, and the like. The program instructions recorded on the media may be those specially designed and constructed for the purposes of the example embodiments, or they may be of the kind well-known and available to those having skill in the computer software arts. Examples of non-transitory computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), a USB, flash memory, and the like. Examples of program instructions include both machine code, such as produced by a compiler, and files containing higher level code that may be executed by the computer using an interpreter. The described hardware devices may be configured to act as one or more software modules to perform the operations of the above-described example embodiments, or vice versa.

While the disclosed invention has been particularly shown and described with reference to exemplary embodiments thereof, it will be understood by one of ordinary skill in the art that various changes in form and details may be made therein without departing from the spirit and scope of the invention as defined by the appended claims. Therefore, it should be understood that the exemplary embodiments described herein should be considered in a descriptive sense only and not for purposes of limitation. The scope of the invention is defined not by the detailed description of the invention but by the appended claims, and all differences within the scope will be construed as being included in the present invention concept.

What is claimed is:

1. A focal point controlling method of a focused ultrasound therapy apparatus, the method comprising:
receiving a plurality of focal point positions on which a focal point of an ultrasonic wave is to be formed;
determining a particle velocity at each of a plurality of elements included in an ultrasound transducer, by reflecting a sound pressure distribution of the ultrasonic wave that is generated by each of the plurality of elements; and
radiating the ultrasonic wave according to the determined particle velocity.

2. The focal point controlling method of claim 1, further comprising receiving a sound pressure to be applied by the ultrasonic wave to each of the plurality of focal point positions.

3. The focal point controlling method of claim 2, wherein the determined particle velocity is a particle velocity required to apply the received sound pressure to each of the plurality of focal point positions.

4. The focal point controlling method of claim 1, wherein the determining of the particle velocity comprises:
calculating, with respect to the plurality of elements and the plurality of focal point positions, a factor indicating a degree to which an ultrasonic wave generated by any one element has an influence on a sound pressure which is applied to any one focal point position;
reflecting the calculated factor to a characteristic equation that indicates a relation between the particle velocity at any one element and the sound pressure which is applied to any one focal point position; and
determining the particle velocity at each of the plurality of elements by using the characteristic equation in which the factor has been reflected.

5. The focal point controlling method of claim 4, wherein the value of the factor has a form of a Gaussian distribution according to an angle between a straight line, which is obtained by connecting a center of gravity of an element with a focal point position, and a cross section in which an ultrasonic wave of the element is generated.

6. The focal point controlling method of claim 4, wherein the factor has a value in the range of 0 to 1, the value of the factor is 1 when a focal point position exists on a straight line that is perpendicular to a cross section in which an ultrasonic wave of the element is generated, from which the ultrasonic wave of the element is generated, and passes through a center of gravity of the element, and the value of the factor is 0 when the focal point position exists on a plane that is obtained by extending the cross section.

7. The focal point controlling method of claim 4, wherein the calculating of the factor comprises calculating a factor with respect to each of the plurality of focal point positions at each of the plurality of elements.

8. The focal point controlling method of claim 1, further comprising:
measuring a temperature of each of the plurality of focal point positions,
wherein the particle velocity at each of the plurality of elements included in the ultrasound transducer is determined based on the temperatures of the plurality of focal point positions.

9. The focal point controlling method of claim 8, wherein the determining of the particle velocity comprises:
determining whether a temperature difference between the plurality of focal point positions is equal to or greater than a predetermined critical value; and
if the temperature difference is equal to or greater than the predetermined critical value, determining a particle velocity that makes the temperature difference between the plurality of focal point positions be less than the predetermined critical value.

10. A non-transitory computer-readable recording medium having recorded thereon a program for executing the method of claim 1.

11. A focused ultrasound therapy apparatus comprising:
an ultrasound transducer to transduce an electrical signal into an ultrasonic wave;
an input unit to receive a plurality of focal point positions on which a focal point of an ultrasonic wave is to be formed; and
a focal point controller to determine a particle velocity at each of a plurality of elements included in the ultrasound transducer, by reflecting a sound pressure distribution of the ultrasonic wave generated by each of the plurality of elements and to control the ultrasound transducer.

12. The focused ultrasound therapy apparatus of claim 11, wherein the input unit receives a sound pressure to be applied by the ultrasonic wave to each of the plurality of focal point positions,
and the particle velocity determined by the focal point controller is a particle velocity required to apply the received sound pressure to each of the plurality of focal point positions.

13. The focused ultrasound therapy apparatus of claim 11, wherein the focal point controller comprises:
a factor calculator to calculate, with respect to the plurality of elements and the plurality of focal point positions, a factor indicating a degree to which an ultrasonic wave generated by any one element has an influence on a sound pressure which is applied to any one focal point position; and
a particle velocity determination unit to determine the particle velocity by reflecting the calculated factor to a characteristic equation which indicates a relation between the particle velocity at any one element and the sound pressure which is applied to any one focal point position.

14. The focused ultrasound therapy apparatus of claim 13, wherein the value of the factor has a form of a Gaussian distribution according to an angle between a straight line, which is obtained by connecting a center of gravity of an element with a focal point position, and a cross section in which an ultrasonic wave of the element is generated.

15. The focused ultrasound therapy apparatus of claim 13, wherein the factor has a value in the range of 0 to 1, the value of the factor is 1 when a focal point position exists on a straight line that is perpendicular to a cross section in which an ultrasonic wave of the element is generated, from which the ultrasonic wave of the element is generated, and passes through a center of gravity of the element, and the value of the factor is 0 when the focal point position exists on a plane that is obtained by extending the cross section.

16. The focused ultrasound therapy apparatus of claim 13, wherein the factor calculator calculates a factor with respect to each of the plurality of focal point positions at each of the plurality of elements.

17. The focused ultrasound therapy apparatus of claim 11, further comprising a feedback unit to measure a temperature of each of the plurality of focal point positions and to feed the measured temperature back to the focal point controller to adjust a particle velocity so that the temperatures of the plurality of focal point positions become uniform.

18. The focused ultrasound therapy apparatus of claim 17, wherein the feedback unit determines whether a temperature difference between the plurality of focal point positions is equal to or greater than a predetermined critical value, and
if the temperature difference is equal to or greater than the predetermined critical value, the focal point controller adjusts the particle velocity to make the temperature difference between the plurality of focal point positions be less than the predetermined critical value.

19. A focal point controlling method of a focused ultrasound therapy apparatus, the method comprising:
controlling sound pressures to be applied to a plurality of focal point positions on which a focal point of an ultrasonic wave is to be formed by determining a particle velocity at each of a plurality of elements included in an ultrasound transducer through reflecting a sound pressure distribution of the ultrasonic wave generated by each of the plurality of elements; and
radiating the ultrasonic wave according to the determined particle velocity.

20. The focal point controlling method of claim 19, further comprising:
controlling temperatures of the plurality of focal point positions on which the focal point of the ultrasonic wave is formed by determining whether a temperature difference between the plurality of focal point positions exceeds a threshold value and by adjusting a particle velocity for each of the plurality of elements based on the determination.
